(19) United States
(12) Patent Application Publication
(10) Pub. No.: US 2002/0094327 A1
(43) Pub. Date: Jul. 18, 2002

(54) Targeting pluripotent stem cells to tissues

(76) Inventor: Bryon E. Petersen, Gainesville, FL (US)

Correspondence Address: Stanley A. Kim, Akerman, Senterfitt & Edison, P.A., 222 Lakeview Avenue, Suite 400, P.O. Box 3188, West Palm Beach, FL, 33402-3188, US

(21) Appl. No.: 10/013,015

(22) Filed: Nov. 5, 2001

Related U.S. Application Data
(60) Provisional application No. 60/246,028, filed on Nov. 5, 2000.

(51) Int. Cl. A61K 48/00; C12N 5/08

(52) U.S. Cl. 424/93.21; 435/370; 514/12

(57) ABSTRACT

A non-naturally occurring method of modulating the targeting a pluripotent stem cell to a target tissue of a mammalian subject from another site in the mammalian subject includes the step of: increasing or decreasing the concentration of SDF-1 alpha protein in the target tissue.

### CROSS REFERENCE TO RELATED APPLICATION

The present application claims the benefit of United States provisional patent application No. 60/246,028, filed Nov. 5, 2000.

### FIELD OF THE INVENTION

The invention relates generally to the fields of developmental biology and medicine. More particularly, the invention relates to compositions and methods for directing stems cells to selected tissues.

### BACKGROUND

Organ failure is a major cause of morbidity and mortality throughout the world. While a variety of drugs have been developed that treat the symptoms of organ failure, in many cases these drugs do not restore the organ to a healthy state, but rather merely ameliorate some of the symptoms of the disease. A theoretically more preferable method to treat organ failure is to simply replace the failed organ itself, e.g., by organ transplantation. Indeed, organ transplantation has proven to be a successful procedure in many cases. Despite this, organ transplantation remains a complex process replete with both clinical and practical problems. Predominant among the clinical problems is the risk of immune system-mediated rejection. Compounding this, a major practical problem is the lack of available donor organs. For example, presently in the United States, the number of patients in need of a replacement organ far exceeds the number of available donor organs.

In view of these problems, a proposed alternative method for treating organ failure is organ regeneration wherein damaged cells of a failing organ are replaced with new, undamaged cells. Organ regeneration is known to occur naturally in some cases of organ damage. For example, in response to a mild insult that causes some hepatocyte death, the remaining, normally quiescent, undamaged hepatocytes are activated to divide to form new cells that restore lost liver function.

While methods for treating organ failure by inducing tissue regeneration are quite promising in theory, several practical problems must be overcome before such methods can be successfully implemented in the clinical setting. A significant problem in this respect is that most mammalian organs lose the ability to regenerate after the fetal or early post-natal stages. Moreover, even among those organs having a limited ability to regenerate in an adult, severe or chronic insults often damage the organs beyond the ability to repair itself.

### SUMMARY

The invention relates to a method for selectively directing migration of pluripotent stem cells to a target tissue within a subject by modulating the level of SDF-1 alpha protein in the target tissue. By increasing the number of pluripotent stem cells that traffic to the target tissue, the rate of tissue repair can be increased because there will be a greater number of pluripotent stem cells in the target tissue that can differentiate into cells which can repopulate and partially or wholly restore the normal function of the damaged tissue. For example, where a liver has been damaged to the point where its hepatocytes cannot replicate in sufficient quantity to restore normal liver function, the levels of SDF-1 alpha protein can be artificially increased (e.g., by intrahepatic injection of the chemokine). The high local concentrations of SDF-1 alpha protein will then cause pluripotent stem cells (e.g., oval cells derived from hematopoietic stem cells) to be recruited and/or retained into the damaged liver at a greater than normal rate. Once in the liver, these pluripotent stems cells can differentiate (with or without the help of other agents such as morphogens) into new hepatocytes to replace the damaged cells and restore liver function.

Accordingly, the invention features a non-naturally occurring method of targeting a pluripotent stem cell to a target tissue of a mammalian subject from another site in the mammalian subject. The method includes the step of increasing the concentration of SDF-1 alpha protein (or a SDF-1 alpha agonist such as a peptidomimetic) in the target tissue. This step can be accomplished by introducing purified SDF-1 alpha protein into the mammalian subject, e.g., by parenteral administration such as intravenous injection, intraarterial injection, injection into the target tissue, or intrahepatic injection where the target tissue is the liver. As an alternative, the step of introducing purified SDF-1 alpha into the mammalian subject can be performed by introducing a matrix impregnated with a SDF-1 alpha protein or agonist into the target tissue (e.g., liver).

Where the target tissue includes a target cell (e.g., a hepatocyte), the step of increasing the concentration of SDF-1 alpha protein in the target tissue can be performed by introducing an expression vector having a nucleic acid encoding an SDF-1 alpha protein into the target cell or introducing into the target cell a purified substance that increases the expression of SDF-1 alpha protein from the target cell (e.g., by increasing the transcription of a gene encoding SDF-1 alpha protein; increasing the translation of an mRNA encoding SDF-1 alpha protein; and/or decreasing the degradation of an mRNA encoding SDF-1 alpha protein). The step of increasing the transcription of a gene encoding SDF-1 alpha can include introducing an exogenous promoter upstream of the gene encoding SDF-1 alpha.

Methods within the invention can further include the step of increasing the number of stem cells in the peripheral blood of the mammalian subject such as by administering an agent that causes a pluripotent stem cell to mobilize from the bone marrow of the animal to the peripheral blood of the animal (e.g., a colony stimulating factor such as G-CSF).

In another aspect, the invention includes a non-naturally occurring method of preventing the recruitment of a pluripotent stem cell to a target tissue of a mammalian subject from another site in the mammalian subject, for example, to reduce undesired addition of new hepatocytes in a liver. This method includes the step of decreasing the binding of a SDF-1 alpha protein to a CXCR4 receptor in the target tissue, e.g., by decreasing the concentration of active SDF-1 alpha in the target tissue or by introducing a purified SDF-1 alpha antagonist into the mammalian subject. Introducing a purified SDF-1 alpha antagonist, can be performed, e.g., by parenteral administration such as intravenous injection, intraarterial injection, injection into the target tissue, or intrahepatic injection. As an alternative, the step of introducing purified SDF-1 alpha antagonist into the mammalian subject can be performed by introducing a matrix impregnated with SDF-1 alpha antagonist into the target tissue (e.g., liver).

Where the target tissue includes a target cell (e.g., a hepatocyte), the step of decreasing the concentration of SDF-1 alpha in the target tissue can be performed by introducing an expression vector having a nucleic acid encoding a SDF-1 alpha antagonist (e.g., an anti-sense nucleic acid or ribozyme that blocks expression of SDF-1 alpha protein) into the target cell or introducing into the target cell a purified substance that decreases the expression of SDF-1 alpha protein from the target cell (e.g., by decreasing the transcription of a gene encoding SDF-1 alpha protein; decreasing the translation of an mRNA encoding SDF-1 alpha protein, and/or increasing the degradation of an mRNA encoding SDF-1 alpha protein). The step of decreasing the transcription of a gene encoding SDF-1 alpha can include introducing an exogenous agent that disrupts the normal function of a promoter upstream of a gene encoding SDF-1 alpha protein.

Methods within the invention can further include the step of decreasing the number of stem cells in the peripheral blood of the mammalian subject such as by administering an agent that prevents a pluripotent stem cell from mobilizing from the bone marrow of the animal to the peripheral blood of the animal (e.g., a colony stimulating factor antagonist).

Unless otherwise defined, all technical terms used herein have the same meaning as commonly understood by one of ordinary skill in the art to which this invention belongs. Commonly understood definitions of molecular biology terms can be found in Rieger et al., Glossary of Genetics: Classical and Molecular, 5th edition, Springer-Verlag: New York, 1991; and Lewin, Genes V, Oxford University Press: New York, 1994.

By the term “gene” is meant a nucleic acid molecule that codes for a particular protein, or in certain cases, a functional or structural RNA molecule. For example, a SDF-1 alpha gene encodes a SDF-1 alpha protein.

As used herein, a “nucleic acid” or a “nucleic acid molecule” means a chain of two or more nucleotides such as RNA (ribonucleic acid) and DNA (deoxyribonucleic acid). A “purified” nucleic acid molecule is one that has been substantially separated or isolated away from other nucleic acid sequences in a cell or organism in which the nucleic acid naturally occurs (e.g., 30, 40, 50, 60, 70, 80, 90, 95, 96, 97, 98, 99, 100% free of contaminants). The term includes, e.g., a recombinant nucleic acid molecule incorporated into a vector, a plasmid, a virus, or a genome of a prokaryote or eukaryote. Examples of purified nucleic acids include cDNAs, fragments of genomic nucleic acids, nucleic acids produced by polymerase chain reaction (PCR), nucleic acids formed by restriction enzyme treatment of genomic nucleic acids, recombinant nucleic acids, and chemically synthesized nucleic acid molecules. A “recombinant” nucleic acid molecule is one made by an artificial combination of two otherwise separated segments of sequence, e.g., by chemical synthesis or by the manipulation of isolated segments of nucleic acids by genetic engineering techniques.

By the terms “SDF-1 alpha gene,” “SDF-1 alpha polynucleotide,” “nucleic acid encoding SDF-1 alpha,” or “SDF-1 alpha nucleic acid” is meant a native or non-native SDF-1 alpha-encoding nucleic acid sequence, e.g., a native SDF-1 alpha cDNA; a nucleic acid having sequences from which SDF-1 alpha CDNA can be transcribed; and/or allelic variants and homologs of the foregoing. The terms encompass double-stranded DNA, single-stranded DNA, and RNA.

As used herein, “protein” or “polypeptide” are used synonymously to mean any peptide-linked chain of amino acids, regardless of length or post-translational modification, e.g., glycosylation or phosphorylation. An “purified” polypeptide is one that has been substantially separated or isolated away from other polypeptides in a cell or organism in which the polypeptide naturally occurs (e.g., 30, 40, 50, 60, 70, 80, 90, 95, 96, 97, 98, 99, 100% free of contaminants).

By the terms “SDF-1 alpha protein” or “SDF-1 alpha polypeptide” is meant an expression product of a SDF-1 alpha gene or a protein that shares at least 65% (but preferably 75, 80, 85, 90, 95, 96, 97 ,98, or 99%) amino acid sequence identity with a native SDF-1 alpha protein and displays a functional activity of the native protein. A “functional activity” of a protein is any activity associated with the physiological function of the protein. For example, a functional activity of a SDF-1 alpha protein is the ability to mediate cellular chemotaxis.

When referring to a nucleic acid molecule or polypeptide, the term “native” refers to a naturally occurring (e.g., a “wild-type”) nucleic acid or polypeptide. A “homolog” of a SDF-1 alpha gene is a gene sequence encoding a SDF-1 alpha polypeptide isolated from an organism other than a mammal. Similarly, a “homolog” of a native SDF-1 alpha polypeptide is an expression product of a SDF-1 alpha homolog.

A “fragment” of a SDF-1 alpha nucleic acid is a portion of a SDF-1 alpha nucleic acid that is less than full-length and comprises at least a minimum length capable of hybridizing specifically with a native SDF-1 alpha nucleic acid under stringent hybridization conditions. The length of such a fragment is preferably at least 15 nucleotides, more preferably at least 20 nucleotides, and most preferably at least 30 nucleotides of a native SDF-1 alpha nucleic acid sequence. A “fragment” of a SDF-1 alpha polypeptide is a portion of a SDF-1 alpha polypeptide that is less than full-length (e.g., a polypeptide consisting of 5, 10, 15, 20, 30, 40, 50, 75, or more amino acids of a native SDF-1 alpha protein), and preferably retains at least one functional activity of a native SDF-1 alpha protein.

When referring to hybridization of one nucleic acid to another, “low stringency conditions” means in 10% formamide, 5× Denhart's solution, 6×SSPE, 0.2% SDS at 42° C., followed by washing in 1×SSPE, 0.2% SDS, at 50° C.; “moderate stringency conditions” means in 50% formamide, 5×Denhart's solution, 5×SSPE, 0.2% SDS at 42° C., followed by washing in 0.2×SSPE, 0.2% SDS, at 65° C.; and “high stringency conditions” means in 50% formamide, 5×Denhart's solution, 5×SSPE, 0.2% SDS at 42° C., followed by washing in 0.1×SSPE, and 0.1% SDS at 65° C. The phrase “stringent hybridization conditions” means low, moderate, or high stringency conditions.

As used herein, “sequence identity” means the percentage of identical subunits at corresponding positions in two sequences when the two sequences are aligned to maximize subunit matching, i.e., taking into account gaps and insertions. When a subunit position in both of the two sequences is occupied by the same monomeric subunit, e.g., if a given position is occupied by an adenine in each of two DNA molecules, then the molecules are identical at that position. For example, if 7 positions in a sequence 10 nucleotides (or amino acids) in length are identical to the corresponding positions in a second 10-nucleotide (or amino acid) sequence, then the two sequences have 70% sequence identity. Preferably, the length of the compared sequences is at least 60 nucleotides (or 20 amino acids), more preferably at least 75 nucleotides (or 25 amino acids), and most preferably 90 nucleotides (or 30 amino acids). Sequence identity is typically measured using sequence analysis software (e.g., Sequence Analysis Software Package of the Genetics Computer Group, University of Wisconsin Biotechnology Center, 1710 University Avenue, Madison, Wis. 53705).

As used herein, the term “vector” refers to a nucleic acid molecule capable of transporting another nucleic acid to which it has been linked. One type of preferred vector is an episome, i.e., a nucleic acid capable of extra-chromosomal replication. A vector capable of directing the expression of a gene to which it is operatively linked is referred to herein as an “expression vector.”

A first nucleic acid sequence is “operably” linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked nucleic acid sequences are contiguous and, where necessary to join two protein coding regions, in reading frame.

By the term “SDF-1 alpha-specific antibody” is meant an antibody that binds a SDF-1 alpha protein, but displays no substantial binding to other naturally occurring proteins other than those sharing the same antigenic determinants as SDF-1 alpha. The term includes polyclonal and monoclonal antibodies.

As used herein, “bind,” “binds,” or “interacts with” means that one molecule recognizes and adheres to a particular second molecule in a sample, but does not substantially recognize or adhere to other structurally unrelated molecules in the sample. Generally, a first molecule that “specifically binds” a second molecule has a binding affinity greater than about 10⁵ to 10⁶ liters/mole for that second molecule.

Although methods and materials similar or equivalent to those described herein can be used in the practice or testing of the present invention, suitable methods and materials are described below. All publications, patent applications, patents, and other references mentioned herein are incorporated by reference in their entirety. In the case of conflict, the present specification, including definitions will control. In addition, the particular embodiments discussed below are illustrative only and not intended to be limiting.

### DETAILED DESCRIPTION

The invention encompasses compositions and methods relating to the use of SDF-1 alpha SDF-1 alpha agonists, or SDF-1 alpha antagonists to modulate the targeting pf pluripotent stem cells to tissues within a subject. The below described preferred embodiments illustrate adaptations of these compositions and methods. Nonetheless, from the description of these embodiments, other aspects of the invention can be made and/or practiced based on the description provided below.

### General Biological Methods

Methods involving conventional molecular biology techniques are described herein. Such techniques are generally known in the art and are described in detail in methodology treatises such as Molecular Cloning: A Laboratory Manual, 2nd ed., vol. 1-3, ed. Sambrook et al., Cold Spring Harbor Laboratory Press, Cold Spring Harbor, N.Y., 1989; and Current Protocols in Molecular Biology, ed. Ausubel et al., Greene Publishing and Wiley-Interscience, New York, 1992 (with periodic updates). Methods for chemical synthesis of nucleic acids are discussed, for example, in Beaucage and Carruthers, Tetra. Letts. 22:1859-1862, 1981, and Matteucci et al., J. Am. Chem. Soc. 103:3185, 1981. Chemical synthesis of nucleic acids can be performed, for example, on commercial automated oligonucleotide synthesizers. Immunological methods (e.g., preparation of antigen-specific antibodies, immunoprecipitation, and immunoblotting) are described, e.g., in Current Protocols in Immunology, ed. Coligan et al., John Wiley & Sons, New York, 1991; and Methods of Immunological Analysis, ed. Masseyeff et al., John Wiley & Sons, New York, 1992. Conventional methods of gene transfer and gene therapy can also be adapted for use in the present invention. See, e.g., Gene Therapy: Principles and Applications, ed. T. Blackenstein, Springer Verlag, 1999; Gene Therapy Protocols (Methods in Molecular Medicine), ed. P. D. Robbins, Humana Press, 1997; and Retro-vectors for Human Gene Therapy, ed. C. P. Hodgson, Springer Verlag, 1996.

### SDF-1 Alpha Proteins

The present invention utilizes a purified SDF-1 alpha protein. The amino acid sequence of a number of different native mammalian SDF-1 alpha proteins are known, including human, rat, mouse, and cat. See, e.g., Shirozu et al., Genomics, 28:495, 1995; Tashiro et al., Science 261:600, 1993; Nishimura et al., Eur. J. Immunogenet. 25:303, 1998; and GenBank Accession No. AF189724. A preferred form of SDF-1 alpha protein is a purified native SDF-1 alpha protein that has an amino acid sequence identical to one of the foregoing mammalian SDF-1 alpha proteins. Variants of native mammalian SDF-1 alpha proteins such as fragments, analogs and derivatives of native mammalian SDF-1 alpha proteins may also be used in the invention. Such variants include, e.g., a polypeptide encoded by a naturally occurring allelic variant of native SDF-1 alpha gene (i.e., a naturally occurring nucleic acid that encodes a naturally occurring mammalian SDF-1 alpha protein), a polypeptide encoded by an alternative splice form of a native SDF-1 alpha gene, a polypeptide encoded by a homolog of a native SDF-1 alpha gene, and a polypeptide encoded by a non-naturally occurring variant of a native SDF-1 alpha gene.

SDF-1 alpha protein variants have a peptide sequence that differs from a native SDF-1 alpha protein in one or more amino acids. The peptide sequence of such variants can feature a deletion, addition, or substitution of one or more amino acids of a native SDF-1 alpha protein. Amino acid insertions are preferably of about 1 to 4 contiguous amino acids, and deletions are preferably of about 1 to 10 contiguous amino acids. In some applications, variant SDF-1 alpha proteins substantially maintain a native SDF-1 alpha protein functional activity (e.g., the ability to cause cellular chemotaxis). For other applications, variant SDF-1 alpha proteins lack or feature a significant reduction in a SDF-1 alpha protein functional activity. Where it is desired to retain a functional activity of native SDF-1 alpha protein, preferred SDF-1 alpha protein variants can be made by expressing nucleic acid molecules within the invention that feature silent or conservative changes. Variant SDF-1 alpha proteins with substantial changes in functional activity can be made by expressing nucleic acid molecules within the invention that feature less than conservative changes.

SDF-1 alpha protein fragments corresponding to one or more particular motifs and/or domains or to arbitrary sizes, for example, at least 5, 10, 25, 50, or 75 amino acids in length are within the scope of the present invention. Isolated peptidyl portions of SDF-1 alpha proteins can be obtained by screening peptides recombinantly produced from the corresponding fragment of the nucleic acid encoding such peptides. In addition, fragments can be chemically synthesized using techniques known in the art such as conventional Merrifield solid phase f-Moc or t-Boc chemistry. For example, a SDF-1 alpha protein of the present invention may be arbitrarily divided into fragments of desired length with no overlap of the fragments, or preferably divided into overlapping fragments of a desired length. The fragments can be produced (recombinantly or by chemical synthesis) and tested to identify those peptidyl fragments which can function as either agonists or antagonists of native SDF-1 alpha protein.

Another aspect of the present invention concerns recombinant forms of the SDF-1 alpha proteins. Recombinant polypeptides preferred by the present invention, in addition to a native SDF-1 alpha protein, are encoded by a nucleic acid that has at least 85% sequence identity (e.g., 85, 86, 87, 88, 89, 90, 91, 92, 93, 94, 95, 96, 97, 98, 99, 100%) with the nucleic acid sequence of a gene encoding a mammalian SDF-1 alpha protein. In a preferred embodiment, variant SDF-1 alpha proteins have one or more functional activities of native SDF-1 alpha protein.

SDF-1 alpha protein variants can be generated through various techniques known in the art. For example, SDF-1 alpha protein variants can be made by mutagenesis, such as by introducing discrete point mutation(s), or by truncation. Mutation can give rise to a SDF-1 alpha protein variant having substantially the same, or merely a subset of the functional activity of native SDF-1 alpha protein. Alternatively, antagonistic forms of the protein can be generated which are able to inhibit the function of a naturally occurring form of the protein, such as by competitively binding to another molecule that interacts with a SDF-1 alpha protein. In addition, agonistic forms of the protein may be generated that constitutively express on or more of the functional activities of a native SDF-1 alpha protein. Other SDF-1 alpha protein variants that can be generated include those that are resistant to proteolytic cleavage, as for example, due to mutations which alter protease target sequences. Whether a change in the amino acid sequence of a peptide results in a variant having one or more functional activities of a native SDF-1 alpha protein can be readily determined by testing the variant for a native SDF-1 alpha protein functional activity.

As another example, SDF-1 alpha protein variants can be generated from a degenerate oligonucleotide sequence. Chemical synthesis of a degenerate gene sequence can be carried out in an automatic DNA synthesizer, and the synthetic genes then ligated into an appropriate expression vector. The purpose of a degenerate set of genes is to provide, in one mixture, all of the sequences encoding the desired set of potential SDF-1 alpha protein sequences. The synthesis of degenerate oligonucleotides is well known in the art (see for example, Narang, SA (1983) Tetrahedron 39:3; Itakura et al. (1981) Recombinant DNA, Proc 3rd Cleveland Sympos. Macromolecules, ed. A G Walton, Amsterdam: Elsevier pp 273-289; Itakura et al. (1984) Annu. Rev. Biochem. 53:323; Itakura et al. (1984) Science 198:1056; Ike et al. (1983) Nucleic Acid Res. 11:477. Such techniques have been employed in the directed evolution of other proteins (see, for example, Scott et al. (1990) Science 249:386-390; Roberts et al. (1992) Proc. Natl. Acad. Sci. USA 89:2429-2433; Devlin et al. (1990) Science 249:404-406; Cwirla et al. (1990) Proc. Natl. Acad. Sci. USA 87:6378-6382; as well as U.S. Pat. Nos. 5,223,409; 5,198,346; and 5,096,815).

Similarly, a library of coding sequence fragments can be provided for a SDF-1 alpha gene clone in order to generate a variegated population of SDF-1 alpha protein fragments for screening and subsequent selection of fragments having one or more native SDF-1 alpha protein functional activities. A variety of techniques are known in the art for generating such libraries, including chemical synthesis. In one embodiment, a library of coding sequence fragments can be generated by (i) treating a double-stranded PCR fragment of a SDF-1 alpha gene coding sequence with a nuclease under conditions wherein nicking occurs only about once per molecule; (ii) denaturing the double-stranded DNA; (iii) renaturing the DNA to form double-stranded DNA which can include sense/antisense pairs from different nicked products; (iv) removing single-stranded portions from reformed duplexes by treatment with S1 nuclease; and (v) ligating the resulting fragment library into an expression vector. By this exemplary method, an expression library can be derived which codes for N-terminal, C-terminal and internal fragments of various sizes.

A wide range of techniques are known in the art for screening gene products of combinatorial libraries made by point mutations or truncation, and for screening cDNA libraries for gene products having a certain property. Such techniques will be generally adaptable for rapid screening of the gene libraries generated by the combinatorial mutagenesis of SDF-1 alpha gene variants. The most widely used techniques for screening large gene libraries typically comprise cloning the gene library into replicable expression vectors, transforming appropriate cells with the resulting library of vectors, and expressing the combinatorial genes under conditions in which detection of a desired activity facilitates relatively easy isolation of the vector encoding the gene whose product was detected.

Combinatorial mutagenesis has a potential to generate very large libraries of mutant proteins, e.g., in the order of 1026 molecules. Combinatorial libraries of this size may be technically challenging to screen even with high throughput screening assays. To overcome this problem, techniques such as recursive ensemble mutagenesis (REM) that allow one to avoid the very high proportion of non-functional proteins in a random library and simply enhance the frequency of functional proteins (thus decreasing the complexity required to achieve a useful sampling of sequence space) can be used. REM is an algorithm which enhances the frequency of functional mutants in a library when an appropriate selection or screening method is employed (Arkin and Yourvan (1992) Proc. Natl. Acad. Sci. USA 89:7811-7815; Yourvan et al. (1992) Parallel Problem Solving from Nature, 2., In Maenner and Manderick, eds., Elsevier Publishing Co., Amsterdam, pp. 401-410; Delgrave et al. (1993) Protein Engineering 6(3):327-331).

The invention also provides for reduction of SDF-1 alpha proteins to generate mimetics, e.g., peptide or non-peptide agents that are able to disrupt binding of a SDF-1 alpha protein to other proteins or molecules with which a native SDF-1 alpha protein interacts. Thus, the mutagenic techniques described can also be used to map which determinants of a SDF-1 alpha protein participate in protein-protein interactions involved in, for example, binding of a SDF-1 alpha protein to other proteins that interact with SDF-1 alpha protein, e.g., its receptor, CXCR4. To illustrate, the critical residues of a SDF-1 alpha protein which are involved in molecular recognition of, for example, a SDF-1 alpha protein receptor such as CXCR4 can be determined and used to generate SDF-1 alpha protein-derived peptidomimetics which competitively inhibit binding of a SDF-1 alpha protein with that moiety. By employing, for example, scanning mutagenesis to map the amino acid residues of a SDF-1 alpha protein that are involved in binding a SDF-1 alpha protein receptor such as CXCR4, peptidomimetic compounds can be generated which mimic those residues of a native SDF-1 alpha protein. Such mimetics may then be used to interfere with the normal function of a SDF-1 alpha protein. For instance, non-hydrolyzable peptide analogs of such residues can be generated using benzodiazepine (e.g., see Freidinger et al. in Peptides: Chemistry and Biology, G. R. Marshall ed., ESCOM Publisher: Leiden, Netherlands, 1988), azepine (e.g., see Huffman et al. in Peptides: Chemistry and Biology, G. R. Marshall ed., ESCOM Publisher: Leiden, Netherlands, 1988), substituted gamma lactam rings (Garvey et al. in Peptides: Chemistry and Biology, G. R. Marshall ed., ESCOM Publisher: Leiden, Netherlands, 1988), keto-methylene pseudopeptides (Ewenson et al. (1986) J. Med. Chem. 29:295; and Ewenson et al. in Peptides: Structure and Function (Proceedings of the 9th American Peptide Symposium) Pierce Chemical Co. Rockland, Ill., 1985), beta-turn dipeptide cores (Nagai et al. (1985) Tetrahedron Lett 26:647; and Sato et al. (1986) J. Chem. Soc. Perkin. Trans. 1:1231), and beta-aminoalcohols (Gordon et al. (1985) Biochem. Biophys. Res. Commun. 126:419; and Dann et al. (1986) Biochem. Biophys. Res. Commun. 134:71). SDF-1 alpha proteins may also be chemically modified to create SDF-1 alpha protein derivatives by forming covalent or aggregate conjugates with other chemical moieties, such as glycosyl groups, lipids, phosphate, acetyl groups and the like. Covalent derivatives of a SDF-1 alpha protein can be prepared by linking the chemical moieties to functional groups on amino acid side chains of the protein or at the N-terminus or at the C-terminus of the protein.

The present invention further pertains to methods of producing the subject SDF-1 alpha proteins. For example, a host cell transfected with a nucleic acid vector directing expression of a nucleotide sequence encoding the subject polypeptides can be cultured under appropriate conditions to allow expression of the peptide to occur. The cells may be harvested, lysed, and the protein isolated. A recombinant SDF-1 alpha protein can be isolated from host cells using techniques known in the art for purifying proteins including ion-exchange chromatography, gel filtration chromatography, ultrafiltration, electrophoresis, and immunoaffinity purification with antibodies specific for such protein.

### SDF-1 Alpha Protein Agonists and Antagonists

Also for use in the invention are molecules that differ from native SDF-1 alpha proteins but still bind to a SDF-1 alpha protein receptor (e.g., CXCR4) or otherwise interfere with the binding of a SDF-1 alpha protein receptor with a SDF-1 alpha protein. These molecules include those that cause signaling through the receptor (e.g., as measured by an increase in chemotaxis) similar to that caused by a native SDF-1 alpha protein (i.e., SDF-1 alpha protein “agonists”), and those that prevent such signaling (i.e., SDF-1 alpha protein “antagonists”). SDF-1 alpha protein agonists can, in some cases, be substituted for a native SDF-1 alpha protein. SDF-1 alpha protein antagonists can be used to block or reduce the function of a native SDF-1 alpha protein, e.g., by competing with a native SDF-1 alpha protein for binding a SDF-1 alpha protein receptor such as CXCR4 or by binding a site on a SDF-1 alpha protein necessary for receptor binding.

As discussed above, a variety of peptide or peptido-mimetic SDF-1 alpha protein agonists or antagonists can be made utilizing conventional techniques. In addition, antibodies or antibody fragments can be made against receptors that bind SDF-1 alpha protein receptors (such as CXCR4), and then screened to identify those that act as agonists or antagonists of a native SDF-1 alpha protein. Still further, SDF-1 alpha protein agonists or antagonists can be identified by screening libraries of other molecules (such as small organic or inorganic molecules) by identifying those that bind SDF-1 alpha protein receptors such as CXCR4 or those that inhibit binding of a SDF-1 alpha protein to a SDF-1 alpha protein receptors. Those identified can be further characterized as agonists or antagonists based on the type of signals they induce or prevent in cells.

### Nucleic Acids

Nucleic acid molecules for use in the invention include those of native genes that encode a mammalian SDF-1 alpha protein and non-native nucleic acids that encode a mammalian SDF-1 alpha protein. Such nucleic acid molecules may be in the form of RNA or in the form of DNA (e.g., cDNA, genomic DNA, and synthetic DNA). The DNA may be double-stranded or single-stranded, and if single-stranded may be the coding (sense) strand or non-coding (anti-sense) strand. The coding sequence which encodes a SDF-1 alpha protein may be identical to a nucleotide sequence shown in U.S. Pat. No. 5,563,048; GenBank Accession No. AF189724, or GenBank Accession No. L120029. It may also be a different coding sequence which, as a result of the redundancy or degeneracy of the genetic code, encodes the same polypeptide as such polynucleotides.

Other nucleic acid molecules within the invention are variants of a native SDF-1 alpha gene such as those that encode fragments, analogs and derivatives of a native SDF-1 alpha protein. Such variants may be, e.g., a naturally occurring allelic variant of a native SDF-1 alpha gene, a homolog of a native SDF-1 alpha gene, or a non-naturally occurring variant of a native SDF-1 alpha gene. These variants have a nucleotide sequence that differs from a native SDF-1 alpha gene in one or more bases. For example, the nucleotide sequence of such variants can feature a deletion, addition, or substitution of one or more nucleotides of a native SDF-1 alpha gene. Nucleic acid insertions are preferably of about 1 to 10 contiguous nucleotides, and deletions are preferably of about 1 to 30 contiguous nucleotides.

In other applications, variant SDF-1 alpha proteins displaying substantial changes in structure can be generated by making nucleotide substitutions that cause less than conservative changes in the encoded polypeptide. Examples of such nucleotide substitutions are those that cause changes in (a) the structure of the polypeptide backbone; (b) the charge or hydrophobicity of the polypeptide; or (c) the bulk of an amino acid side chain. Nucleotide substitutions generally expected to produce the greatest changes in protein properties are those that cause non-conservative changes in codons. Examples of codon changes that are likely to cause major changes in protein structure are those that cause substitution of (a) a hydrophilic residue, e.g., serine or threonine, for (or by) a hydrophobic residue, e.g., leucine, isoleucine, phenylalanine, valine or alanine; (b) a cysteine or proline for (or by) any other residue; (c) a residue having an electropositive side chain, e.g., lysine, arginine, or histidine, for (or by) an electronegative residue, e.g., glutamine or aspartine; or (d) a residue having a bulky side chain, e.g., phenylalanine, for (or by) one not having a side chain, e.g., glycine.

Naturally occurring allelic variants of a native SDF-1 alpha gene within the invention are nucleic acids isolated from mammalian tissue that have at least 75% (e.g., 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, and 99%) sequence identity with a native SDF-1 alpha gene, and encode polypeptides having structural similarity to a native SDF-1 alpha protein. Homologs of a native SDF-1 alpha gene within the invention are nucleic acids isolated from other species that have at least 75% (e.g., 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, and 99%) sequence identity with the native gene, and encode polypeptides having structural similarity to a native SDF-1 alpha protein. Public and/or proprietary nucleic acid databases can be searched to identify other nucleic acid molecules having a high percent (e.g., 70, 80, 90% or more) sequence identity to a native SDF-1 alpha gene.

Non-naturally occurring SDF-1 alpha gene variants are nucleic acids that do not occur in nature (e.g., are made by the hand of man), have at least 75% (e.g., 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, and 99%) sequence identity with a native SDF-1 alpha gene, and encode polypeptides having structural similarity to a native SDF-1 alpha protein. Examples of non-naturally occurring SDF-1 alpha gene variants are those that encode a fragment of a native SDF-1 alpha protein, those that hybridize to a native SDF-1 alpha gene or a complement of to a native SDF-1 alpha gene under stringent conditions, those that share at least 65% sequence identity with a native SDF-1 alpha gene or a complement of a native SDF-1 alpha gene, and those that encode a SDF-1 alpha fusion protein.

Nucleic acids encoding fragments of a native SDF-1 alpha protein within the invention are those that encode, e.g., 2, 5, 10, 20, 30, 40, 50, 60, 70 or more amino acid residues of a native SDF-1 alpha protein. Shorter oligonucleotides (e.g., those of 6, 7, 8, 9, 10, 11, 12, 13, 14, 15, 16, 17, 18, 19, 20, 30, 40, 50, 60, 70, 80, 90, or 100 base pairs in length) that encode or hybridize with nucleic acids that encode fragments of a native SDF-1 alpha protein can be used as probes, primers, or antisense molecules. Longer polynucleotides (e.g., those of 125, 150, 175, 200, 225, or 250 base pairs) that encode or hybridize with nucleic acids that encode fragments of a native SDF-1 alpha protein can also be used in various aspects of the invention. Nucleic acids encoding fragments of a native SDF-1 alpha protein can be made by enzymatic digestion (e.g., using a restriction enzyme) or chemical degradation of the full length native SDF-1 alpha gene or variants thereof.

Nucleic acids that hybridize under stringent conditions to one of the foregoing nucleic acids can also be used in the invention. For example, such nucleic acids can be those that hybridize to one of the foregoing nucleic acids under low stringency conditions, moderate stringency conditions, or high stringency conditions are within the invention.

Nucleic acid molecules encoding a SDF-1 alpha fusion protein may also be used in the invention. Such nucleic acids can be made by preparing a construct (e.g., an expression vector) that expresses a SDF-1 alpha fusion protein when introduced into a suitable host. For example, such a construct can be made by ligating a first polynucleotide encoding a SDF-1 alpha protein fused in frame with a second polynucleotide encoding another protein such that expression of the construct in a suitable expression system yields a fusion protein.

The oligonucleotides of the invention can be DNA or RNA or chimeric mixtures or derivatives or modified versions thereof, single-stranded or double-stranded. Such oligonucleotides can be modified at the base moiety, sugar moiety, or phosphate backbone, for example, to improve stability of the molecule, hybridization, etc. Oligonucleotides within the invention may additionally include other appended groups such as peptides (e.g., for targeting host cell receptors in vivo), or agents facilitating transport across the cell membrane (see, e.g., Letsinger et al. (1989) Proc. Natl. Acad. Sci. U.S.A. 86:6553-6556; Lemaitre et al. (1987) Proc. Natl. Acad. Sci. USA 84:648-652; PCT Publication No. WO 88/09810, published Dec. 15, 1988), hybridization-triggered cleavage agents. (See, e.g., Krol et al. (1988) BioTechniques 6:958-976) or intercalating agents. (See, e.g, Zon (1988) Pharm. Res. 5:539-549). To this end, the oligonucleotides may be conjugated to another molecule, e.g., a peptide, hybridization triggered cross-linking agent, transport agent, hybridization-triggered cleavage agent, etc.

### Probes and Primers

Detection of SDF-1 alpha expression can be performed using oligonucleotide probes (i.e., isolated nucleic acid molecules conjugated with a detectable label or reporter molecule, e.g., a radioactive isotope, ligand, chemiluminescent agent, or enzyme); and oligonucleotide primers (i.e., isolated nucleic acid molecules that can be annealed to a complementary target DNA strand by nucleic acid hybridization to form a hybrid between the primer and the target DNA strand, then extended along the target DNA strand by a polymerase, e.g., a DNA polymerase). Primer pairs can be used for amplification of a nucleic acid sequence, e.g., by the polymerase chain reaction (PCR) or other conventional nucleic-acid amplification methods. Probes and primers within the invention are generally 15 nucleotides or more in length, preferably 20 nucleotides or more, more preferably 25 nucleotides, and most preferably 30 nucleotides or more. Preferred probes and primers are those that hybridize to a native SDF-1 alpha gene sequence under high stringency conditions, and those that hybridize to SDF-1 alpha gene homologs under at least moderately stringent conditions. Preferably, probes and primers according to the present invention have complete sequence identity with a native SDF-1 alpha gene sequence, although probes differing from the native gene sequence and that retain the ability to hybridize to native SDF-1 alpha gene sequences under stringent conditions may be designed by conventional methods. Primers and probes based on a native SDF-1 alpha gene sequence disclosed herein can be used to confirm (and, if necessary, to correct) a disclosed native SDF-1 alpha gene sequence by conventional methods, e.g., by re-cloning and sequencing a native SDF-1 alpha cDNA.

### SDF-1 Alpha-Specific Antibodies

SDF-1 alpha proteins and/or SDF-1 alpha protein receptors such as CXCR4 (or immunogenic fragments or analogs thereof) can be used to raise antibodies useful in the invention. Such proteins can be produced by recombinant techniques or synthesized as described above. In general, SDF-1 alpha proteins can be coupled to a carrier protein, such as KLH, as described in Ausubel et al., supra, mixed with an adjuvant, and injected into a host mammal. Antibodies produced in that animal can then be purified by peptide antigen affinity chromatography. In particular, various host animals can be immunized by injection with a SDF-1 alpha protein or an antigenic fragment thereof. Commonly employed host animals include rabbits, mice, guinea pigs, and rats. Various adjuvants that can be used to increase the immunological response depend on the host species and include Freund's adjuvant (complete and incomplete), mineral gels such as aluminum hydroxide, surface active substances such as lysolecithin, pluronic polyols, polyanions, peptides, oil emulsions, keyhole limpet hemocyanin, and dinitrophenol. Other potentially useful adjuvants include BCG (bacille Calmette-Guerin) and Corynebacterium parvum.

Polyclonal antibodies are heterogeneous populations of antibody molecules that are contained in the sera of the immunized animals. Antibodies within the invention therefore include polyclonal antibodies and, in addition, monoclonal antibodies, single chain antibodies, Fab fragments, F(ab′)2 fragments, and molecules produced using a Fab expression library. Monoclonal antibodies, which are homogeneous populations of antibodies to a particular antigen, can be prepared using one of the SDF-1 alpha proteins described above and standard hybridoma technology (see, for example, Kohler et al., Nature 256:495, 1975; Kohler et al., Eur. J. Immunol. 6:511, 1976; Kohler et al., Eur. J. Immunol. 6:292, 1976; Hammerling et al., “Monoclonal Antibodies and T Cell Hybridomas,” Elsevier, N.Y., 1981; Ausubel et al., supra). In particular, monoclonal antibodies can be obtained by any technique that provides for the production of antibody molecules by continuous cell lines in culture such as described in Kohler et al., Nature 256:495, 1975, and U.S. Pat. No. 4,376,110; the human B-cell hybridoma technique (Kosbor et al., Immunology Today 4:72, 1983; Cole et al., Proc. Natl. Acad. Sci. USA 80:2026, 1983), and the EBV-hybridoma technique (Cole et al., “Monoclonal Antibodies and Cancer Therapy,” Alan R. Liss, Inc., pp. 77-96, 1983). Such antibodies can be of any immunoglobulin class including IgG, IgM, IgE, IgA, IgD and any subclass thereof. A hybridoma producing a mAb of the invention may be cultivated in vitro or in vivo. The ability to produce high titers of mAbs in vivo makes this a particularly useful method of production.

Once produced, polyclonal or monoclonal antibodies can be tested for specific SDF-1 alpha protein (or SDF-1 alpha protein receptor) recognition by Western blot or immunoprecipitation analysis by standard methods, for example, as described in Ausubel et al., supra. Antibodies that specifically recognize and bind to a SDF-1 alpha protein (or SDF-1 alpha protein receptor) are useful in the invention. For example, such antibodies can be used in an immunoassay to monitor the level of SDF-1 alpha protein produced by a mammal (e.g., to determine the amount or location of SDF-1 alpha protein in a diseased tissue such as a liver).

Preferably, SDF-1 alpha protein (or SDF-1 alpha protein receptor) selective antibodies of the invention are produced using fragments of a SDF-1 alpha protein (or SDF-1 alpha protein receptor) that lie outside highly conserved regions and appear likely to be antigenic, by criteria such as high frequency of charged residues. Cross-reactive anti-SDF-1 alpha protein (or SDF-1 alpha protein receptor) antibodies are produced using a fragment of SDF-1 alpha protein that is conserved amongst members of this family of proteins. In one specific example, such fragments are generated by standard techniques of PCR, and are then cloned into the pGEX expression vector (Ausubel et al., supra). Fusion proteins are expressed in E. coli and purified using a glutathione agarose affinity matrix as described in Ausubel, et al., supra.

In some cases it may be desirable to minimize the potential problems of low affinity or specificity of antisera. In such circumstances, two or three fusions can be generated for each protein, and each fusion can be injected into at least two rabbits. Antisera can be raised by injections in a series, preferably including at least three booster injections. Antiserum is also checked for its ability to immunoprecipitate recombinant SDF-1 alpha proteins or control proteins, such as glucocorticoid receptor, CAT, or luciferase.

The antibodies of the invention can be used, for example, in the detection of a SDF-1 alpha protein (or SDF-1 alpha protein receptor) in a biological sample, e.g., a liver section or cell. Antibodies also can be used in a screening assay to measure the effect of a candidate compound on expression or localization of SDF-1 alpha protein or SDF-1 alpha protein receptor. Additionally, such antibodies can be used to interfere with the interaction of a SDF-1 alpha protein and other molecules that bind the SDF-1 alpha protein such as a SDF-1 alpha protein receptor.

Techniques described for the production of single chain antibodies (e.g., U.S. Pat. Nos. 4,946,778, 4,946,778, and 4,704,692) can be adapted to produce single chain antibodies against a SDF-1 alpha protein, or a fragment thereof. Single chain antibodies are formed by linking the heavy and light chain fragments of the Fv region via an amino acid bridge, resulting in a single chain polypeptide.

Antibody fragments that recognize and bind to specific epitopes can be generated by known techniques. For example, such fragments include but are not limited to F(ab′)2 fragments that can be produced by pepsin digestion of the antibody molecule, and Fab fragments that can be generated by reducing the disulfide bridges of F(ab′)2 fragments. Alternatively, Fab expression libraries can be constructed (Huse et al., Science 246:1275, 1989) to allow rapid and easy identification of monoclonal Fab fragments with the desired specificity.

### Detection of SDF-1 Alpha Polynucleotides and Proteins

The invention encompasses methods for detecting the presence of a SDF-1 alpha protein or a SDF-1 alpha nucleic acid in a biological sample as well as methods for measuring the level of a SDF-1 alpha protein or a SDF-1 alpha nucleic acid in a biological sample. An exemplary method for detecting the presence or absence of a SDF-1 alpha protein or a SDF-1 alpha nucleic acid in a biological sample involves obtaining a biological sample from a test subject (e.g., a human patient), contacting the biological sample with a compound or an agent capable of detecting a SDF-1 alpha protein or a SDF-1 alpha nucleic acid (e.g., mRNA or genomic DNA) in a biological sample, and analyzing binding of the compound or agent to the sample after washing. Those samples that specifically bind the compound or agent express a SDF-1 alpha protein or a SDF-1 alpha nucleic acid.

Detection methods of the invention can be used to detect an mRNA encoding a SDF-1 alpha protein, a genomic DNA encoding a SDF-1 alpha protein, or a SDF-1 alpha protein in a biological sample in vitro as well as in vivo. For example, in vitro techniques for detection of mRNAs encoding a SDF-1 alpha protein include Northern hybridizations and in situ hybridizations. In vitro techniques for detection of a SDF-1 alpha protein include enzyme linked immunosorbent assays (ELISAs), Western blots, immunoprecipitations and immunofluorescence. In vitro techniques for detection of genomic DNA encoding a SDF-1 alpha protein include Southern hybridizations. Furthermore, in vivo techniques for detection of a SDF-1 alpha protein include introducing a labeled anti-SDF-1 alpha antibody into a biological sample or test subject. For example, the antibody can be labeled with a radioactive or radiopaque marker whose presence and location in a biological sample or test subject can be detected by standard imaging techniques.

### Mammalian Subjects, Target Tissues, Target Cells, and Stem Cells

The invention provides methods involving modulating levels of SDF-1 alpha in a target tissue of a mammalian subject. Mammalian subjects include any mammal such as human beings, rats, mice, cats, dogs, goats, sheep, horses, monkeys, apes, rabbits, cattle, etc. The mammalian subject can be in any stage of development including adults, young animals, and neonates. Mammalian subjects also include those in a fetal stage of development. Target tissues can be any within the mammalian subject such as liver, kidney, heart, lungs, components of gastrointestinal tract, pancreas, gall bladder, urinary bladder, the central nervous system including the brain, skin, bones, etc. Target cells for use in the invention can include any cell in or that migrates to a target tissue, e.g., an oval cell or a hepatocyte. Pluripotent stem cells described in the invention are any cells that can be induced to differentiate into another cell type. Examples include hematopoietic stem cells that can differentiate into oval cells, and oval cells or other cells that can differentiate into hepatocytes.

### Stem Cell Mobilization

The invention provides methods involving increasing or decreasing the rate of stem cell movement from the bone marrow to the peripheral blood (i.e., “stem cell mobilization”). A number of substances are known to increase or decrease this rate. Each of these may be used in the invention. For example, to increase the number of stem cells in the peripheral blood of a mammalian subject, an agent that causes a pluripotent stem cell to mobilize from the bone marrow can be administered to the subject. A number of such agents are known. See, e.g., those described in International Application WO 00/50048; and colony stimulating factors such as G-CSF. Similarly, to decrease the number of stem cells in the peripheral blood of a mammalian subject, an agent that prevents a pluripotent stem cell to mobilize from the bone marrow can be administered to the subject. For example, an antibody against a colony stimulating factors such as G-CSF can be employed.

### Delivery and Expression of SDF-1 Alpha

The invention provides methods for both increasing the level (e.g., concentration) of SDF-1 alpha protein in a target tissue and decreasing the binding of a SDF-1 alpha protein to a SDF-1 alpha protein receptor. Both methods can be achieved in a variety of ways including, for example, administering SDF-1 alpha protein, SDF-1 alpha protein agonist, or SDF-1 alpha protein antagonist to the mammalian subject. Such compositions of the invention may be administered to the mammalian subject neat or in pharmaceutically acceptable carriers (e.g., physiological saline) in a manner selected on the basis of mode and route of administration and standard pharmaceutical practice. A list of pharmaceutically acceptable carriers, as well as pharmaceutical formulations, can be found in Remington's Pharmaceutical Sciences, a standard text in this field, and in USP/NF. Other substances may be added to the compositions to stabilize and/or preserve the compositions. For example, glycine, maltose, and/or thimerosal or other preservative may be added to the compositions.

Rather than introducing a purified SDF-1 alpha protein, agonist, or antagonist to a mammalian subject, the level of SDF-1 alpha protein can be regulated by introducing a purified substance into a target cell that modulates expression of SDF-1 alpha protein in the target tissue. For example, an expression vector encoding a SDF-1 alpha protein can be introduced into a cell making up the target tissue. Transcription of the insert encoding the SDF-1 alpha protein would then lead to production of SDF-1 alpha protein in that cell. In a similar manner, SDF-1 alpha protein expression in a target cell can be reduced by introducing into that cell an expression vector encoding an anti-sense nucleic acid or ribozyme that inhibits expression of SDF-1 alpha protein.

Natural or synthetic nucleic acids according to the present invention can be incorporated into recombinant nucleic acid constructs, typically DNA constructs, capable of introduction into and replication in a target cell. Such a construct preferably is a vector that includes a replication system and sequences that are capable of transcription and translation of a polypeptide-encoding sequence in a given target cell. For the practice of the present invention, conventional compositions and methods for preparing and using vectors and host cells are employed, as discussed, e.g., in Sambrook et al., 1989, or Ausubel et al., 1992.

Various other techniques could also be used in the invention to modulate SDF-1 alpha levels in a target tissue. For example, agents that increase the transcription of a gene encoding SDF-1 alpha; increase the translation of an mRNA encoding SDF-1 alpha, and/ or those that decrease the degradation of an mRNA encoding SDF-1 alpha could be used to increase SDF-1 alpha protein levels. In the same manner, agents that decrease the transcription of a gene encoding SDF-1 alpha; decrease the translation of an mRNA encoding SDF-1 alpha, and/or those that increase the degradation of an mRNA encoding SDF-1 alpha could be used to decrease SDF-1 alpha protein levels. Modulating the rate of transcription from a gene within a cell can be accomplished by introducing an exogenous promoter upstream of the gene encoding SDF-1 alpha. For example, the wild-type promoter can be interrupted to reduce the rate of transcription or replaced to increase the rate of transcription (e.g., using a strong promoter). This can be performed by, e.g., adapting known techniques that utilize site specific homologous recombination. See, e.g., U.S. Pat. Nos. 5,641,670; 5,733,761; 5,968,502; 6,048,729; 6,054,288; and 6,063,630. Enhancer elements which facilitate expression of a heterologous gene may also be employed.

Expression of SDF-1 alpha nucleic acids in a cell can also be modulated using anti-sense nucleic acids or ribozymes. Use of an antisense nucleic acid involves the designing of oligonucleotides (either DNA or RNA) that are complementary to a portion of a selected mRNA encoding the protein to be downregulated. These oligonucleotides bind to complementary mRNA transcripts and prevent their translation.

Oligonucleotides that are complementary to the 5′ end of the message, for example, the 5′ untranslated sequence up to and including the AUG initiation codon, should work most efficiently at inhibiting translation. However, sequences complementary to the 3′ untranslated sequences of mRNAs have also been shown to be effective at inhibiting translation of mRNAs as well (Wagner, Nature 372:333, 1984). Thus, oligonucleotides complementary to either the 5′ or 3′ non-translated, non-coding regions of a SDF-1 alpha nucleic acid, could be used in an antisense approach to inhibit translation of endogenous SDF-1 alpha-mRNA. Oligonucleotides complementary to the 5′ untranslated region of the mRNA should include the complement of the AUG start codon.

Antisense oligonucleotides complementary to mRNA coding regions are less efficient inhibitors of translation but could be used in accordance with the invention. Whether designed to hybridize to the 5′, 3′, or coding region of SDF-1 alpha mRNA, antisense nucleic acids should be at least six nucleotides in length, and are preferably oligonucleotides ranging from 6 to about 50 nucleotides in length. In specific aspects, the oligonucleotide is at least 10 nucleotides, at least 17 nucleotides, at least 25 nucleotides, or at least 50 nucleotides.

Regardless of the choice of target sequence, as with other therapeutic strategies directed to SDF-1 alpha, it is preferred that in vitro studies are first performed to assess the ability of an antisense oligonucleotide to inhibit gene expression. If desired, the assessment can be quantitative. It is preferred that these studies utilize controls that distinguish between antisense gene inhibition and any nonspecific biological effect that an oligonucleotide may cause. It is also preferred that these studies compare levels of the target RNA or protein with that of an internal control RNA or protein. Additionally, it is envisioned that results obtained using an antisense oligonucleotide are compared with those obtained using a control oligonucleotide. Preferably, the control oligonucleotide is of approximately the same length as the test oligonucleotide, and the nucleotide sequence of the control oligonucleotide differs from that of the test antisense sequence no more than is necessary to prevent specific hybridization between the control oligonucleotide and the targeted RNA sequence.

The oligonucleotides can contain DNA or RNA, or they can contain chimeric mixtures, derivatives, or modified versions thereof that are either single-stranded or double-stranded. The oligonucleotide can be modified at the base moiety, sugar moiety, or phosphate backbone, for example, to improve stability of the molecule, hybridization, etc. Modified sugar moieties can be selected from the group including, but not limited to, arabinose, 2-fluoroarabinose, xylulose, and hexose. A modified phosphate backbone can be selected from the group consisting of a phosphorothioate, a phosphorodithioate, a phosphoramidothioate, a phosphoramidate, a phosphordiamidate, a methylphosphonate, an alkyl phosphotriester, and a formacetal, or an analog of any of these backbones.

The oligonucleotide can include other appended groups such as peptides (e.g., for disrupting the transport properties of the molecule in host cells in vivo), or agents that facilitate transport across the cell membrane (as described, for example, in Letsinger et al., Proc. Natl. Acad. Sci. USA 86:6553, 1989; Lemaitre et al., Proc. Natl. Acad. Sci. USA 84:648, 1987; PCT Publication No. WO 88/09810) or the blood-brain barrier (see, for example, PCT Publication No. WO 89/10134), or hybridization-triggered cleavage agents (see, for example, Krol et al., BioTechniques 6:958, 1988), or intercalating agents (see, for example, Zon, Pharm. Res. 5:539, 1988). To this end, the oligonucleotide can be conjugated to another molecule, for example, a peptide, a hybridization triggered cross-linking agent, a transport agent, or a hybridization-triggered cleavage agent.

An antisense oligonucleotide can comprise at least one modified base moiety that is selected from the group including, but not limited to, 5-fluoro-uracil, 5-bromouracil, 5-chlorouracil, 5-iodouracil, hypoxanthine, xantine, 4-acetylcytosine, 5-(carboxyhydroxyl-methyl) uracil, 5-carboxymethylaminomethyl-2-thiouridine, 5-carboxymethyl-aminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methyl-aminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-theouracil, 2-thiouracil, 4-thio-uracil, 5-methyluracil, uracil-5-oxyacetic acid methylester, uracil-5-oxyacetic acid (v), 5-methyl-2-thiouracil, 2-(3-amino-3-N-2-carboxypropl) uracil, (acp3)w, and 2,6-diaminopurine.

In yet another embodiment, the antisense oligonucleotide is an alpha-anomeric oligonucleotide. An alpha-anomeric oligonucleotide forms specific double-stranded hybrids with complementary RNA in which, contrary to the usual beta-units, the strands run parallel to each other (Gautier et al., Nucl. Acids. Res. 15:6625, 1987). The oligonucleotide is a 2′-O-methylribonucleotide (Inoue et al., Nucl. Acids Res. 15:6131, 1987), or a chimeric RNA-DNA analog (Inoue et al., FEBS Lett. 215:327, 1987).

Antisense oligonucleotides of the invention can be synthesized by standard methods known in the art, for example, by use of an automated DNA synthesizer (such as are commercially available from Biosearch, Applied Biosystems, etc.). As examples, phosphorothioate oligonucleotides can be synthesized by the method of Stein et al. (Nucl. Acids Res. 16:3209, 1988), and methylphosphonate oligonucleotides can be prepared by use of controlled pore glass polymer supports (Sarin et al., Proc. Natl. Acad. Sci. USA 85:7448, 1988).

For therapeutic application, antisense molecules of the invention should be delivered to cells that express SDF-1 alpha in vivo. A number of methods have been developed for delivering antisense DNA or RNA to cells; for example, antisense molecules can be injected directly into the tissue site. Alternatively, modified antisense molecules, which are designed to target cells that express SDF-1 alpha (e.g., antisense molecules linked to peptides or antibodies that specifically bind receptors or antigens expressed on the target cell surface) can be administered systemically. However, it is often difficult to achieve intracellular concentrations of antisense molecules that are sufficient to suppress translation of endogenous mRNAs. Therefore, a preferred approach uses a recombinant DNA construct in which the antisense oligonucleotide is placed under the control of a strong pol III or pol II promoter. The use of such a construct to transfect target cells in the patient will result in the transcription of sufficient amounts of single stranded RNAs that will form complementary base pairs with endogenous SDF-1 alpha transcripts and thereby prevent translation of SDF-1 alpha mRNA. For example, a vector can be introduced in vivo in such a way that it is taken up by a cell and thereafter directs the transcription of an antisense RNA. Such a vector can remain episomal or become chromosomally integrated, as long as it can be transcribed to produce the desired antisense RNA.

Vectors encoding a SDF-1 alpha antisense sequence can be constructed by recombinant DNA technology methods that are standard practice in the art. Suitable vectors include plasmid vectors, viral vectors, or other types of vectors known or newly discovered in the art. The criterion for use is only that the vector be capable of replicating and expressing the SDF-1 alpha antisense molecule in mammalian cells. Expression of the sequence encoding the antisense RNA can be directed by any promoter known in the art to act in mammalian, and preferably in human, cells. Such promoters can be inducible or constitutively active and include, but are not limited to: the SV40 early promoter region (Bernoist et al., Nature 290:304, 1981); the promoter contained in the 3′ long terminal repeat of Rous sarcoma virus (Yamamoto et al., Cell 22:787-797, 1988); the herpes thymidine kinase promoter (Wagner et al., Proc. Natl. Acad. Sci. USA 78:1441, 1981); or the regulatory sequences of the metallothionein gene (Brinster et al., Nature 296:39, 1988).

Ribozyme molecules designed to catalytically cleave SDF-1 alpha mRNA transcripts also can be used to prevent translation of SDF-1 alpha mRNA and expression of SDF-1 alpha polypeptides (see, for example, PCT Publication WO 90/11364; Saraver et al., Science 247:1222, 1990). While various ribozymes that cleave mRNA at site-specific recognition sequences can be used to destroy SDF-1 alpha mRNAs, the use of hammerhead ribozymes is preferred. Hammerhead ribozymes cleave mRNAs at locations dictated by flanking regions that form complementary base pairs with the target mRNA. See, Haseloff et al., Nature 334:585, 1988.

As in the antisense approach, the ribozymes can be composed of modified oligonucleotides (e.g., for improved stability, targeting, etc.), and should be delivered to cells which express the SDF-1 alpha in vivo. A preferred method of delivery involves using a DNA construct “encoding” the ribozyme under the control of a strong constitutive pol III or pol II promoter, so that transfected cells will produce sufficient quantities of the ribozyme to destroy endogenous SDF-1 alpha messages and inhibit translation.

### Viral Vectors

Various techniques using viral vectors for the introduction of a SDF-1 alpha nucleic acid into a cell may be utilized in the methods of the invention. Preferred viral vectors for use in the invention are those that exhibit low toxicity to a host cell and induce production of therapeutically useful quantities of SDF-1 alpha protein in a tissue-specific manner. Viral vector methods and protocols that may be used in the invention are reviewed in Kay et al. Nature Medicine 7:33-40, 2001. The use of specific vectors, including those based on adenoviruses, adeno-associated viruses, herpes viruses, and retroviruses are described in more detail below.

The use of recombinant adenoviruses as gene therapy vectors is discussed in W. C. Russell, Journal of General Virology 81:2573-2604, 2000; and Bramson et al., Curr. Opin. Biotechnol. 6:590-595, 1995. Adenovirus vectors are preferred for use in the invention because they (1) are capable of highly efficient gene expression in target cells and (2) can accommodate a relatively large amount of heterologous (non-viral) DNA. A preferred form of recombinant adenovirus is a “gutless, “high-capacity”, or “helper-dependent” adenovirus vector. Such a vector features, for example, (1) the deletion of all or most viral-coding sequences (those sequences encoding viral proteins), (2) the viral inverted terminal repeats (ITRs) which are sequences required for viral DNA replication, (3) up to 28-32 kb of “exogenous” or “heterologous” sequences (e.g., sequences encoding a SDF-1 alpha protein), and (4) the viral DNA packaging sequence which is required for packaging of the viral genomes into infectious capsids. For specifically targeting liver, preferred variants of such recombinant adenoviral vectors contain tissue-specific (e.g., liver) enhancers and promoters operably linked to a SDF-1 alpha gene.

Other viral vectors that might be used in the invention are adeno-associated virus (AAV)-based vectors. AAV-based vectors are advantageous because they exhibit high transduction efficiency of target cells and can integrate into the host genome in a site-specific manner. Use of recombinant AAV vectors is discussed in detail in Tal, J., J. Biomed. Sci. 7:279-291, 2000 and Monahan and Samulski, Gene Therapy 7:24-30, 2000. A preferred AAV vector comprises a pair of AAV inverted terminal repeats which flank at least one cassette containing a tissue (e.g., liver)—or cell (e.g., hepatocyte)—specific promoter operably linked to a SDF-1 alpha nucleic acid. The DNA sequence of the AAV vector, including the ITRs, the promoter and SDF-1 alpha gene may be integrated into the host genome.

The use of herpes simplex virus (HSV)-based vectors is discussed in detail in Cotter and Robertson, Curr. Opin. Mol. Ther. 1:633-644, 1999. HSV vectors deleted of one or more immediate early genes (IE) are advantageous because they are generally non-cytotoxic, persist in a state similar to latency in the host cell, and afford efficient host cell transduction. Recombinant HSV vectors can incorporate approximately 30 kb of heterologous nucleic acid. A preferred HSV vector is one that: (1) is engineered from HSV type I, (2) has its IE genes deleted, and (3) contains a tissue-specific (e.g., liver) promoter operably linked to a SDF-1 alpha nucleic acid. HSV amplicon vectors may also be useful in various methods of the invention. Typically, HSV amplicon vectors are approximately 15 kb in length, and possess a viral origin of replication and packaging sequences.

Retroviruses such as C-type retroviruses and lentiviruses might also be used in the invention. For example, retroviral vectors may be based on murine leukemia virus (MLV). See, e.g., Hu and Pathak, Pharmacol. Rev. 52:493-511, 2000 and Fong et al., Crit. Rev. Ther. Drug Carrier Syst. 17:1-60, 2000. MLV-based vectors may contain up to 8 kb of heterologous (therapeutic) DNA in place of the viral genes. The heterologous DNA may include a tissue-specific promoter and a SDF-1 alpha nucleic acid. In methods of delivery to a liver, it may also encode a ligand to a liver cell-specific receptor.

Additional retroviral vectors that might be used are replication-defective lentivirus-based vectors, including human immunodeficiency (HIV)-based vectors. See, e.g., Vigna and Naldini, J. Gene Med. 5:308-316, 2000 and Miyoshi et al., J. Virol. 72:8150-8157, 1998. Lentiviral vectors are advantageous in that they are capable of infecting both actively dividing and non-dividing cells. They are also highly efficient at transducing human epithelial cells. HIV vectors are preferred for targeting liver tissue as they have been shown to efficiently infect hepatic cells. Lentiviral vectors for use in the invention may be derived from human and non-human (including SIV) lentiviruses. Preferred lentiviral vectors include nucleic acid sequences required for vector propagation as well as a tissue-specific promoter (e.g., liver) operably linked to a SDF-1 alpha gene. These former may include the viral LTRs, a primer binding site, a polypurine tract, att sites, and an encapsidation site.

A lentiviral vector may be packaged into any suitable lentiviral capsid. The substitution of one particle protein with another from a different virus is referred to as “pseudotyping”. The vector capsid may contain viral envelope proteins from other viruses, including murine leukemia virus (MLV) or vesicular stomatitis virus (VSV). The use of the VSV G-protein yields a high vector titer and results in greater stability of the vector virus particles.

Alphavirus-based vectors, such as those made from semliki forest virus (SFV) and sindbis virus (SIN), might also be used in the invention. Use of alphaviruses is described in Lundstrom, K., Intervirology 43:247-257, 2000 and Perri et al., Journal of Virology 74:9802-9807, 2000. Alphavirus vectors typically are constructed in a format known as a replicon. A replicon may contain (1) alphavirus genetic elements required for RNA replication, and (2) a heterologous nucleic acid such as one encoding a SDF-1 alpha nucleic acid. Within an alphivirus replicon, the heterologous nucleic acid may be operably linked to a tissue-specific (e.g., liver) promoter or enhancer.

Recombinant, replication-defective alphavirus vectors are advantageous because they are capable of high-level heterologous (therapeutic) gene expression, and can infect a wide host cell range. Alphavirus replicons may be targeted to specific cell types (e.g., hepatocytes) by displaying on their virion surface a functional heterologous ligand or binding domain that would allow selective binding to target cells expressing a cognate binding partner. Alphavirus replicons may establish latency, and therefore long-term heterologous nucleic acid expression in a host cell. The replicons may also exhibit transient heterologous nucleic acid expression in the host cell. A preferred alphavirus vector or replicon is non-cytopathic.

In many of the viral vectors compatible with methods of the invention, more than one promoter can be included in the vector to allow more than one heterologous gene to be expressed by the vector. Further, the vector can comprise a sequence which encodes a signal peptide or other moiety which facilitates the secretion of a SDF-1 alpha gene product from the host cell.

To combine advantageous properties of two viral vector systems, hybrid viral vectors may be used to deliver a SDF-1 alpha nucleic acid to a target tissue (e.g., liver). Standard techniques for the construction of hybrid vectors are well-known to those skilled in the art. Such techniques can be found, for example, in Sambrook, et al., In Molecular Cloning: A laboratory manual. Cold Spring Harbor, N.Y. or any number of laboratory manuals that discuss recombinant DNA technology. Double-stranded AAV genomes in adenoviral capsids containing a combination of AAV and adenoviral ITRs may be used to transduce cells. In another variation, an AAV vector may be placed into a “gutless”, “helper-dependent” or “high-capacity” adenoviral vector. Adenovirus/AAV hybrid vectors are discussed in Lieber et al., J. Virol. 73:9314-9324, 1999. Retrovirus/adenovirus hybrid vectors are discussed in Zheng et al., Nature Biotechnol. 18:176-186, 2000. Retroviral genomes contained within an adenovirus may integrate within the host cell genome and effect stable SDF-1 alpha gene expression.

Other nucleotide sequence elements which facilitate expression of the SDF-1 alpha gene and cloning of the vector are further contemplated. For example, the presence of enhancers upstream of the promoter or terminators downstream of the coding region, for example, can facilitate expression. In the vectors of the present invention, the presence of elements which enhance liver-specific expression of SDF-1 alpha may be useful for gene therapy.

### Non-viral Delivery

In addition to viral vector-based methods, non-viral methods may also be used to introduce a SDF-1 alpha gene into a host cell. A review of non-viral methods of gene delivery is provided in Nishikawa and Huang, Human Gene Ther. 12:861-870, 2001. A preferred non-viral gene delivery method according to the invention employs plasmid DNA to introduce a SDF-1 alpha nucleic acid into a cell. Plasmid-based gene delivery methods are generally known in the art and are described in references such as Ilan, Y., Curr. Opin. Mol. Ther. 1:116-120, 1999, Wolff, J. A., Neuromuscular Disord. 7:314-318, 1997 and Arztl, Z., Fortbild Qualitatssich 92:681-683, 1998. In a particularly preferred variation of the latter method, plasmid DNA encoding both a SDF-1 alpha protein and an immunomodulatory protein (e.g., a cytokine or fibronectin) is used. Because soluble fibronectin is thought to enhance SDF-1 alpha-induced migration of cells, the delivery of both SDF-1 alpha and fibronectin should increase mobilization of stem cells into the liver.

Methods involving physical techniques for introducing a SDF-1 alpha nucleic acid into a host cell can be adapted for use in the present invention. For example, the particle bombardment method of gene transfer utilizes an Accell device (gene gun) to accelerate DNA-coated microscopic gold particles into target tissue, e.g., the liver. See, e.g., Yang et al., Mol. Med. Today 2:476-481 1996 and Davidson et al., Rev. Wound Repair Regen. 6:452-459, 2000. As another example, cell electropermeabilization (also termed cell electroporation) may be employed to deliver SDF-1 alpha nucleic acids into cells. See, e.g., Preat, V., Ann. Pharm. Fr. 59:239-244 2001.

Synthetic gene transfer molecules can be designed to form multimolecular aggregates with plasmid DNA (e.g., harboring a SDF-1 alpha coding sequence operably linked to a liver-specific promoter). These aggregates can be designed to bind to a target cell (e.g., hepatocyte) surface in a manner that triggers endocytosis and endosomal membrane disruption. In a preferred embodiment for targeting hepatocytes, polymeric DNA-binding cations (including polylysine, protamine, and cationized albumin) are linked to hepatocyte-specific targeting ligands that trigger receptor-mediated endocytosis into hepatocytes. See, e.g., Guy et al., Mol. Biotechnol. 3:237-248, 1995 and Garnett, M. C., Crit. Rev. Ther. Drug Carrier Syst. 16:147-207, 1999. Cationic amphiphiles, including lipopolyamines and cationic lipids, may be used to provide receptor-independent SDF-1 alpha nucleic acid transfer into target cells (e.g., hepatocytes). In addition, preformed cationic liposomes or cationic lipids may be mixed with plasmid DNA to generate cell-transfecting complexes. Methods involving cationic lipid formulations are reviewed in Felgner et al., Ann. N.Y. Acad. Sci. 772:126-139, 1995 and Lasic and Templeton, Adv. Drug Delivery Rev. 20:221-266, 1996. For gene delivery, DNA may also be coupled to an amphipathic cationic peptide (Fominaya et al., J. Gene Med. 2:455-464, 2000).

Methods that involve both viral and non-viral based components may be used according to the invention. For example, an Epstein Barr virus (EBV)-based plasmid for therapeutic gene delivery is described in Cui et al., Gene Therapy 8:1508-1513, 2001. Additionally, a method involving a DNA/ligand/polycationic adjunct coupled to an adenovirus is described in Curiel, D. T., Nat. Immun. 13:141-164, 1994.

In various methods of the invention liver-specific ligands are used to target liver cells. For example, peptides or polypeptides may be used as ligands to target delivery of DNA and proteins to liver-specific receptors. Complexes of protein and ligand or plasmid DNA and ligand mediate preferential protein and DNA transfer into liver cells. As a specific example, the hepatic asialoglycoprotein receptor (ASGPr) binds asialoorosomucoid-polylysine-DNA (ASOR-PL-DNA) complexes and allows target delivery to hepatocytes. Merwin et al., Bioconjug. Chem. 5:612-620.

Methods involving ultrasound contrast agent delivery vehicles may be used in the invention. See, e.g., Newman et al., Echocardiography 18:339-347, 2001 and Lewin et al. Invest. Radiol. 36:9-14, 2001. For instance, a liver cell-targeting moiety may be conjugated to a contrast agent vehicle to result in site-specific (e.g., liver) SDF-1 alpha nucleic acid expression. Gene-bearing microbubbles, which cavitate upon exposure to ultrasound, might be used to deliver the gene to a specific target tissue.

A natural or synthetic matrix that provides support for the delivered agent (e.g., SDF-1 alpha nucleic acid or protein) prior to delivery might be used in the invention. See, for example, the techniques described in Murphy and Mooney, J. Period Res., 34:413-9, 1999 and Vercruysse and Prestwich, Crit. Rev. Ther. Drug Carrier Syst., 15:513-55, 1998. Matrices suitable for use in the invention may be formed from both natural or synthetic materials and may be designed to allow for sustained release of the therapeutic agent and growth factors over prolonged periods of time. For implantation into an animal subject, a preferred matrix is resorbable and/or biocompatible (i.e., does not produce an adverse or allergic reaction when administered to the recipient host). In some embodiments of the invention, matrices are impregnated with growth factors capable of stimulating the chemotaxis and mobilization of stem cells.

DNA microencapsulation may be used to facilitate delivery of a SDF-1 alpha nucleic acid. Microencapsulated gene delivery vehicles may be constructed from low viscosity polymer solutions that are forced to phase invert into fragmented spherical polymer particles when added to appropriate nonsolvents. Methods involving microparticles are discussed in Hsu et al., J. Drug Target 7:313-323, 1999 and Capan et al., Pharm. Res. 16:509-513, 1999.

Methods involving microencapsulated recombinant cells may be used in the invention. Such an approach may be used in either in vivo or ex vivo techniques. Cells that contain an expression vector coding for SDF-1 alpha or that have been engineered to stably express SDF-1 alpha may be encapsulated in microcapsules that provide protection from immune mediators and allow appropriate release of the SDF-1 alpha protein. Preferred microencapsulation particles, also referred to as encapsulation devices, consist of biocompatible and biodegradable components. Techniques involving microencapsulated cells are discussed in Ross et al. Hum. Gen. Ther. 11:2117-2127, 2000 and Fong et al., Crit. Rev. Ther. Drug Carrier Syst. 17:1-60, 2000.

Protein transduction offers an alternative to gene therapy for the delivery of therapeutic proteins into target cells, and methods involving protein transduction are within the scope of the invention. Protein transduction is the internalization of proteins into a host cell from the external environment. The internalization process relies on a protein or peptide which is able to penetrate the cell membrane. To confer this ability on a normally nontransducing protein, the non-transducing protein can be fused to a transduction-mediating protein such as the antennapedia peptide, the HIV TAT protein transduction domain, or the herpes simplex virus VP22 protein. See Ford et al., Gene Ther. 8:1-4, 2001.

### Methods for Gene/Protein/Cell transfer Ex Vivo

The invention provides methods involving ex vivo delivery of a SDF-1 alpha nucleic acid or protein. In a preferred method, hepatocyte-mediated ex vivo gene therapy is used wherein SDF-1 alpha expressing host hepatocytes are introduced into a host liver. A suitable ex vivo protocol includes the steps of harvesting a segment of liver from a donor subject; transfecting a SDF-1 alpha nucleic acid into the harvested hepatocytes; and then transplanting the transfected cells into a host (e.g., the donor subject). A SDF-1 alpha nucleic acid transfected into cells may be operably linked to any suitable regulatory sequence, including a liver-specific promoter and enhancer. Several approaches may be used for delivering transfected hepatocytes into the host, including catheter-mediated delivery into the inferior mesenteric vein, the umbilical vein or the spleen. Autologous and allogeneic cell transplantation is preferred. However, where the donor and host are histoincompatible, the SDF-1 alpha-expressing cells modified ex vivo may be microencapsulated prior to introduction into the host.

### Duration of SDF-1 alpha expression

SDF-1 alpha nucleic acids of the present invention may be expressed for any suitable length of time within the host cell, including transient expression and stable, long-term expression. In a preferred embodiment, the SDF-1 alpha nucleic acid will be expressed in therapeutic amounts for a suitable and defined length of time. Episomally replicating vectors may be used to achieve transient expression, while vectors that integrate chromosomally may be used to achieve long-term expression of a SDF-1 alpha nucleic acid.

### Targeting SDF-1 Alpha Expression to the Liver and Regulating

### SDF-1 Alpha Protein Concentration in Target Tissues

A preferred method of the invention provides for the delivery and expression of a SDF-1 alpha nucleic acid or protein in a tissue-specific (e.g., liver) manner. A number of methods exist for targeting gene delivery and expression to the liver, which are well-known to those skilled in the art. Methods for targeting gene delivery to the liver have been discussed above, and include liver cell receptor-targeting (receptor-mediated uptake systems). In a preferred embodiment, the vectors utilized to deliver a SDF-1 alpha nucleic acid/protein contain a transcriptional promoter to effect liver or hepatocyte-specific SDF-1 alpha expression. Examples of liver-specific promoters include the following: albumin promoter, human C-reactive protein gene promoter, human apolipoprotein B promoter, human apolipoprotein A-I promoter, and human apolipoprotein A-II promoter. In addition, liver-specific enhancers may be incorporated in the vectors, including the human apo E enhancer.

### Effective Doses

A therapeutically effective amount is an amount which is capable of producing a medically desirable result in a treated animal or human. As is well known in the medical arts, dosage for any one animal or human depends on many factors, including the subject's size, body surface area, age, the particular composition to be administered, sex, time and route of administration, general health, and other drugs being administered concurrently. It is expected that an appropriate dosage for intrahepatic administration of purified SDF-1 alpha protein would be in the range of about 0.001 to 10 mg/kg body weight. More specific dosages for purified SDF-1 alpha protein as well as other compositions described herein (e.g., proteins, nucleic acids, or small molecules) can be determined by the method described below.

Toxicity and therapeutic efficacy of the compositions of the invention can be determined by standard pharmaceutical procedures, using cells in culture and/or experimental animals to determine the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compositions that exhibit large therapeutic indices are preferred. While compositions that exhibit toxic side effects may be used, care should be taken to design a delivery system that targets such compounds to the site of the infection or tissues to be treated in order to minimize potential damage to uninvolved tissue and thereby reduce side effects.

The data obtained from cell culture assays and animal studies can be used in formulating a range of dosage for use in humans. The dosage of such compounds lies preferably within the range of circulating concentrations that include an ED₅₀ with little or no toxicity. The dosage may vary within this range depending on the dosage form employed and the route of administration utilized.

### EXAMPLES

The present invention is further illustrated by the following specific examples. The examples are provided for illustration only and are not to be construed as limiting the scope or content of the invention in any way.

### Example 1

Liver Regeneration Models

Bone marrow cells have previously been shown to develop into hepatic oval cells in rodent models. See, e.g., Petersen et al., Science, 284:1168, 1999. Under certain conditions, oval cells have also been shown to differentiate into hepatocytes. Grisham and Thorgeirsson, in Stem Cells, C. S. Potten, Ed., chapter 8, Academic Press: San Diego, 1997; Thorgeirsson et al., Proc. Soc. Exp. Bio., 204:253, 1993; and N. Fausto, in The Liver: Biology and Pathobiology, I. M. Arias et al., Eds., (Raven: New York), 1994. Techniques involving rodent models are described in detail in published international patent application no. WO 00/50048.

### Example 2

SDF-1 alpha expression in models of hepatic injury

Oval cells have been shown not to play an important role in normal liver regeneration brought on by CCl₄ poisoning or partial hepatectomy. In addition, it has been shown that in order to fully activate hepatic oval cells to proliferate, an inhibitory factor, such as 2-AAF (2-acetylaminofluorene), should be employed. Western blot analyses of protein levels of SDF-1 alpha and actin (as a control) in whole cell lysate as well as the membrane fraction of hepatocytes from two different types of liver regeneration models were performed. Hepatic injury was induced by administering CCl₄ to the test animals. Protein of (a) whole cell liver lysate and (b) the membrane fraction of the cell lysate were obtained from rats exposed to an acute dose of CCl₄ alone and CCl₄+2-AAF at various time points following hepatic injury (12h, 24h, 36h, 48h, 72h, 96h, 120h for CCl₄ alone; and 1d, 3d, 5d, 9d, 11d, 13d, and 15d for CCl₄+2-AAF). Approximately 10 ug of protein (pooled samples, n=3) was first electrophoresed on an 8-18% gradient glycine gel to separate the proteins, and then transferred on to a nylon membrane. The membranes were subjected to Western blot analysis using antibodies against SDF-1 alpha and actin (as a control).

Over the time period examined no upregulation of SDF-1 alpha was noted in either the whole cell lysate or membrane fraction samples from the animals treated with CCl₄ alone. However, in the samples where the oval cell compartment was activated (i.e., those where the hepatocytes were exposed to 2-AAF followed by CCl₄ injury), upregulation of SDF-1 alpha was observed from the earliest time period measured (1d post-treatment) to the latest time period measured (15d post-treatment). This upregulation appeared to peak about day 9—the time point coinciding with the approximate peak of oval cell proliferation.

In other experiments, liver injury was induced using (a) acute AA (allyl alcohol) poisoning alone, (b) AA poisoning+2-AAF (which induces a very modest oval cell response), or (c) partial hepatectomy+2-AAF (which induces an oval cell response somewhat less than the CCl₄+2-AAF protocol); and SDF-1 alpha expression was examined by Western blot analyses of the membrane fractions of whole liver cell lysates as described above. Consistent with the very modest oval cell response observed in this model, no upregulation of SDF-1 alpha was observed at any of the time points measured in either of the AA protocols. In contrast, although expression appeared to be less intense than that obtained with the CCl₄+2-AAF protocol, an upregulation of SDF-1 alpha expression was observed in the partial hepatectomy+2-AAF protocol at the earliest time period measured post-treatment (day 5), appeared to peak at day 9, then decrease at 11 days, and further decrease at 13 days (a time period reported to correlate with the differentiation of oval cells into hepatocytes).

### Example 3

Immunohistochemistry

Expression of CXCR4 (the receptor for SDF-1 alpha) on oval cells was examined by immunohistochemistry of rat liver sections using anti-CXCR4 antibodies. Frozen liver tissue sections from rats subjected to (a) the 2-AAF/partial hepatectomy protocol and(b) the 2-AAF/CCl₄ protocol were examined for CXCR4 via peroxidase immuno-staining utilizing the DAB detection system with hematoxylin as a counter stain. In both groups, oval cells that were positive for CXCR4 were clearly identifiable on photomicrographs of the stained sections. Hepatocytes in the same sections (which are negative for CXCR4) did not stain for CXCR4.

In further experiments, liver cells from rats subjected to (a) the 2-AAF/partial hepatectomy protocol and (b) the 2-AAF/CCl₄ protocol were subjected to sorting to purify the oval cells therein (hepatic oval cells express the hematopoietic stem cell marker thy-1). The thy-1+ sorted cells were cytocentrifugated (100,000/slide) and then examined for CXCR4 expression by alkaline phosphatase immuno-staining (using the Vector Blue detection system). Photomicrographs of these preparations showed that purified cells from both groups also clearly stained for CXCR4.

### Other Embodiments

While the above specification contains many specifics, these should not be construed as limitations on the scope of the invention, but rather as examples of preferred embodiments thereof. Many other variations are possible. For example, although much of the foregoing describes the methods for targeting pluripotent stem cells to liver, other tissues could also be targeted by modulating their SDF-12 alpha levels. The step of modulating the level of SDF-1 alpha protein in a target tissue could also be combined with a step of introducing a morphogen into the target tissue to facilitate differentiation of the targeted stem cell into a desired cell type. See, e.g., U.S. Pat. No. 5,849,686. Accordingly, the scope of the invention should be determined not by the embodiments illustrated, but by the appended claims and their legal equivalents.

What is claimed is:

1. A non-naturally occurring method of targeting a pluripotent stem cell to a target tissue of a mammalian subject from another site in the mammalian subject, the method comprising the step of:
increasing the concentration of a SDF-1 alpha protein or SDF-1 alpha agonist in the target tissue.

2. The method of claim 1, wherein the step of increasing the concentration of the SDF-1 alpha protein or agonist in the target tissue includes a step of introducing a purified SDF-1 alpha protein into the mammalian subject.

3. The method of claim 2, wherein the step of introducing the purified SDF-1 alpha protein into the mammalian subject is performed by parenteral administration.

4. The method of claim 3, wherein the parenteral administration is intravenous or intraarterial injection.

5. The method of claim 2, wherein the step of introducing the purified SDF-1 alpha protein into the mammalian subject is performed by injection into the target tissue.

6. The method of claim 5, wherein the target tissue is the mammalian subject's liver and the parenteral administration is intrahepatic injection.

7. The method of claim 2, wherein the step of introducing the purified SDF-1 alpha into the mammalian subject is performed by introducing a matrix impregnated with the SDF-1 alpha protein into the target tissue.

8. The method of claim 7, wherein the target tissue is the mammalian subject's liver.

9. The method of claim 1, wherein the target tissue comprises a target cell and the step of increasing the concentration of the SDF-1 alpha protein or agonist in the target tissue comprises introducing an expression vector having a nucleic acid encoding the SDF-1 alpha protein into the target cell.

10. The method of claim 1, wherein the target tissue comprises a target cell and the step of increasing the concentration of the SDF-1 alpha protein or agonist in the target tissue comprises introducing into the target cell a purified substance that increases the expression of the SDF-1 alpha protein from the target cell.

11. The method of claim 10, wherein the increase in expression of the SDF-1 alpha protein from the target cell is effected by a step selected from the group of:
(a) increasing the transcription of a gene encoding the SDF-1 alpha protein;
(b) increasing the translation of an mRNA encoding the SDF-1 alpha protein, and
(c) decreasing the degradation of an mRNA encoding the SDF-1 alpha protein.

12. The method of claim 11, wherein the increase in expression of the SDF-1 alpha protein from the target cell is effected by step (a) of increasing the transcription of a gene encoding the SDF-1 alpha protein.

13. The method of claim 12, wherein the step of increasing the transcription of a gene encoding the SDF-1 alpha protein comprises introducing an exogenous promoter upstream of the gene encoding the SDF-1 alpha protein.

14. The method of claim 1, further comprising the step of increasing the number of pluripotent stem cells in the peripheral blood of the mammalian subject.

15. The method of claim 14, wherein the step of increasing the number of pluripotent stem cells in the peripheral blood of the mammalian subject comprises administering an agent that causes a pluripotent stem cell to mobilize from the bone marrow of the animal to the peripheral blood of the animal.

16. The method of claim 15, wherein the agent that causes a pluripotent stem cell to mobilize from the bone marrow of the animal to the peripheral blood of the animal is a colony stimulating factor.

17. A non-naturally occurring method of preventing the recruitment of a pluripotent stem cell to a target tissue of a mammalian subject from another site in the mammalian subject, the method comprising the step of:
decreasing the binding of a SDF-1 alpha protein to a CXC4 receptor in the target tissue.

18. The method of claim 1, wherein the step of decreasing the binding of the SDF-1 alpha protein to the CXC4 receptor in the target tissue includes a step of introducing a purified SDF-1 alpha antagonist into the mammalian subject.

19. The method of claim 18, wherein the step of introducing the purified SDF-1 alpha antagonist into the mammalian subject is performed by parenteral administration.

20. The method of claim 19, wherein the parenteral administration is intravenous or intraarterial injection.

21. The method of claim 18, wherein the step of introducing the purified SDF-1 alpha antagonist into the mammalian subject is performed by injection into the target tissue.

22. The method of claim 22, wherein the target tissue is the mammalian subject's liver and the parenteral administration is intrahepatic injection.

23. The method of claim 18, wherein the step of introducing the purified SDF-1 alpha antagonist into the mammalian subject is performed by introducing a matrix impregnated with the SDF-1 alpha antagonist into the target tissue.

24. The method of claim 23, wherein the target tissue is the mammalian subject's liver.

25. The method of claim 17, wherein the target tissue comprises a target cell and the step of decreasing the concentration of the SDF-1 alpha protein in the target tissue comprises introducing an expression vector having a nucleic acid encoding a SDF-1 alpha antagonist into the target cell.

26. The method of claim 17, wherein the target tissue comprises a target cell and the step of decreasing the concentration of the SDF-1 alpha protein in the target tissue comprises introducing into the target cell a purified substance that decreases the expression of the SDF-1 alpha protein from the target cell.

27. The method of claim 26, wherein the decrease in expression of the SDF-1 alpha protein from the target cell is effected by a step selected from the group of:
(a) decreasing the transcription of a gene encoding the SDF-1 alpha protein;
(b) decreasing the translation of an mRNA encoding the SDF-1 alpha protein, and
(c) increasing the degradation of an mRNA encoding the SDF-1 alpha protein.

28. The method of claim 27, wherein the decrease in expression of the SDF-1 alpha protein from the target cell is effected by step (a) of decreasing the transcription of a gene encoding the SDF-1 alpha protein.

29. The method of claim 28, wherein the step of decreasing the transcription of a gene encoding the SDF-1 alpha protein comprises introducing a nucleic acid that disrupts the endogenous promoter of the gene encoding SDF-1 alpha protein.

30. The method of claim 17, further comprising the step of decreasing the number of stem cells in the peripheral blood of the mammalian subject.
